(19) United States
(12) Patent Application Publication
(10) Pub. No.: US 2025/0352049 A1
(43) Pub. Date: Nov. 20, 2025

(54) MEDICAL DEVICE, MEDICAL SYSTEM, METHOD OF OPERATING MEDICAL DEVICE, AND COMPUTER-READABLE RECORDING MEDIUM

(71) Applicant: OLYMPUS MEDICAL SYSTEMS CORP., Tokyo (JP)

(72) Inventors: Yasuo TANIGAMI, Tokyo (JP); Yusuke OTSUKA, Yokohama-shi (JP); Noriko KURODA, Tokyo (JP); Takaaki IGARASHI, Tokyo (JP); Nanako SATO, Sagamihara-shi (JP)

(73) Assignee: OLYMPUS MEDICAL SYSTEMS CORP., Tokyo (JP)

(21) Appl. No.: 19/289,503

(22) Filed: Aug. 4, 2025

Related U.S. Application Data
(63) Continuation of application No. PCT/JP2023/004454, filed on Feb. 9, 2023.

(51) Int. Cl. A61B 1/04; A61B 1/00; A61B 1/12; A61B 1/307

(52) U.S. Cl. CPC A61B 1/043; A61B 1/00087; A61B 1/126; A61B 1/307

(57) ABSTRACT

A medical device includes a processor including hardware, the processor being configured to acquire a first fluorescence image obtained by imaging a target region and a second fluorescence image obtained by imaging the target region after a time at which the first fluorescence image is captured, generate a drive signal for causing a display device to display discharge information indicating a discharge status of a resected piece resected by a resection treatment tool in the target region based on first information included in the first fluorescence image and second information included in the second fluorescence image, and output the drive signal.

### CROSS REFERENCES TO RELATED APPLICATIONS

This application is a continuation of International Application No. PCT/JP2023/004454, filed on Feb. 9, 2023, the entire contents of which are incorporated herein by reference.

### BACKGROUND

### 1. Technical Field

The present disclosure relates to a medical device, a medical system, a method of operating a medical device, and a computer-readable recording medium.

### 2. Related Art

Hitherto, in the medical field, a technology for visualizing a state of cauterization of a subject such as a biological tissue using an energy device or the like is known (see, for example, WO 2020/054723 A). In the technology, the subject is irradiated with excitation light, and an image and information including fluorescence image data generated based on an imaging signal acquired by imaging fluorescence generated from a thermally invasive region of the subject by receiving the excitation light are displayed, thereby visualizing the cauterization state for a user such as an operator.

### SUMMARY

In some embodiments, a medical device includes a processor including hardware, the processor being configured to acquire a first fluorescence image obtained by imaging a target region and a second fluorescence image obtained by imaging the target region after a time at which the first fluorescence image is captured, generate a drive signal for causing a display device to display discharge information indicating a discharge status of a resected piece resected by a resection treatment tool in the target region based on first information included in the first fluorescence image and second information included in the second fluorescence image, and output the drive signal.

In some embodiments, a medical system includes: a light source device including a light source configured to emit excitation light for exciting advanced glycation end products generated by performing a heat treatment on a target region of a biological tissue; an imaging device including an imaging element configured to generate a first fluorescence image obtained by imaging the target region by imaging fluorescence emitted by the excitation light and a second fluorescence image obtained by imaging the target region after a time at which the first fluorescence image is captured; and a medical device including a processor including hardware, the processor being configured to acquire the first fluorescence image and the second fluorescence image, generate a drive signal for causing a display device to display discharge information indicating a discharge status of a resected piece resected by a resection treatment tool in the target region based on first information included in the first fluorescence image and second information included in the second fluorescence image, and output the drive signal.

In some embodiments, provided is a method of operating a medical device including a processor and driven according to a cleaning state of a target region. The method includes: acquiring, by the processor, a first fluorescence image obtained by imaging the target region and a second fluorescence image obtained by imaging the target region after a time at which the first fluorescence image is captured; generating, by the processor, a drive signal for causing a display device to display discharge information indicating a discharge status of a resected piece resected by a resection treatment tool in the target region based on first information included in the first fluorescence image and second information included in the second fluorescence image; and outputting, by the processor, the drive signal.

In some embodiments, provided is a non-transitory computer-readable recording medium with an executable program stored thereon. The program causes a processor of a medical device driven according to a cleaning state of a target region to execute: acquiring a first fluorescence image obtained by imaging the target region and a second fluorescence image obtained by imaging the target region after a time at which the first fluorescence image is captured; generating a drive signal for causing a display device to display discharge information indicating a discharge status of a resected piece resected by a resection treatment tool in the target region based on first information included in the first fluorescence image and second information included in the second fluorescence image; and outputting the drive signal.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a configuration of an endoscope system according to a first embodiment;

FIG. 2 is a block diagram illustrating a functional configuration of a main part of the endoscope system according to the first embodiment;

FIG. 3 is a diagram schematically illustrating wavelength characteristics of excitation light emitted by two light source units according to the first embodiment;

FIG. 4 is a diagram schematically illustrating a configuration of a pixel unit according to the first embodiment;

FIG. 5 is a diagram schematically illustrating a configuration of a color filter according to the first embodiment;

FIG. 6 is a diagram schematically illustrating sensitivity and a wavelength band of each filter according to the first embodiment;

FIG. 7A is a diagram schematically illustrating signal values of G pixels of an imaging element according to the first embodiment;

FIG. 7B is a diagram schematically illustrating signal values of R pixels of the imaging element according to the first embodiment;

FIG. 7C is a diagram schematically illustrating signal values of B pixels of the imaging element according to the first embodiment;

FIG. 8 is a diagram schematically illustrating a configuration of a cut filter according to the first embodiment;

FIG. 9 is a diagram schematically illustrating a transmission characteristic of the cut filter according to the first embodiment;

FIG. 10 is a flowchart illustrating an outline of processing executed by a control device according to the first embodiment;

FIG. 11 is a flowchart illustrating an outline of processing executed by a control device according to a second embodiment;

FIG. 12 is a diagram illustrating a schematic configuration of an endoscope system according to a third embodiment; and

FIG. 13 is a block diagram illustrating a functional configuration of a medical device according to the third embodiment.

### DETAILED DESCRIPTION

Hereinafter, modes for carrying out the present disclosure will be described in detail with reference to the drawings. Note that the present disclosure is not limited to the following embodiments. In addition, each drawing referred to in the following description merely schematically illustrates a shape, a size, and a positional relationship to the extent that the content of the present disclosure can be understood. That is, the present disclosure is not limited only to the shape, the size, and the positional relationship illustrated in each drawing. Further, in the description of the drawings, the same reference signs denote the same parts. Furthermore, as an example of an endoscope system according to the present disclosure, an endoscope system including a rigid endoscope and a medical imaging device will be described.

### First Embodiment

### Configuration of Endoscope System

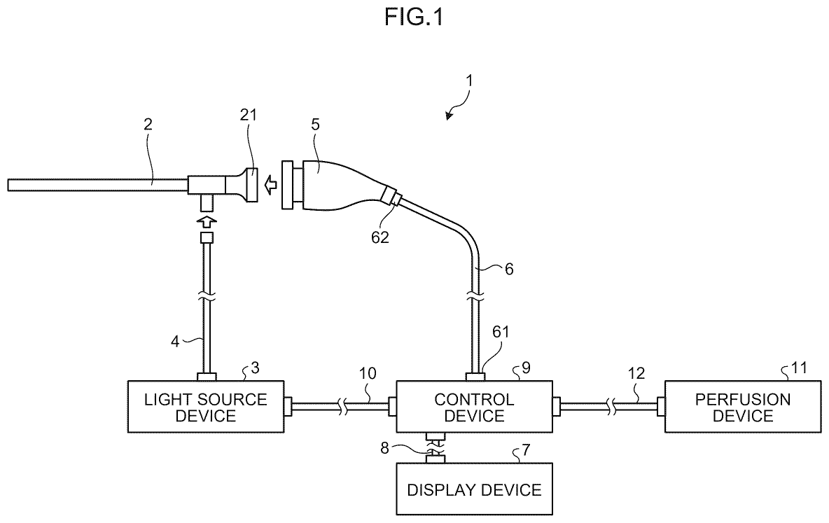

FIG. 1 is a diagram illustrating a configuration of an endoscope system according to a first embodiment. An endoscope system 1 illustrated in FIG. 1 is a system that is used in a medical field and observes and treats a biological tissue in a subject such as a living body. In the first embodiment, a rigid endoscope system using a rigid endoscope (insertion unit 2) illustrated in FIG. 1 will be described as the endoscope system 1, but the present disclosure is not limited thereto, and for example, an endoscope system including a flexible endoscope may be used. Furthermore, an endoscope system can also be applied as the endoscope system 1 to a medical microscope, a medical surgical robot system, or the like that includes a medical imaging device that images a subject and performs surgery, treatment, or the like while displaying an observation image based on an imaging signal (image data) captured by the medical imaging device on a display device.

In addition, in recent years, in the medical field, minimally invasive treatment using an endoscope, a laparoscope, or the like has been widely performed. For example, as the minimally invasive treatment using an endoscope, a laparoscope, or the like, endoscopic submucosal dissection (ESD), laparoscopy and endoscopy cooperative surgery (LECS), non-exposed endoscopic wall-inversion surgery (NEWS), transurethral resection of the bladder tumor (TUR-bt), or the like is widely performed. In the minimally invasive treatment, for example, in order to mark a region to be operated as pretreatment when performing treatment, an operator such as a doctor performs resection by cauterization, marking treatment by heat treatment, or the like on a region of interest (pathogenic region) having an affected part in the biological tissue using a treatment tool of an energy device that emits high-frequency, ultrasonic, or microwave energy. In addition, also in actual treatment, the operator performs treatment such as resection and coagulation of the biological tissue of the subject by using the energy device or the like. Therefore, the endoscope system 1 illustrated in FIG. 1 is used when performing the surgery or treatment on the subject using the treatment tool (not illustrated) of the energy device or the like capable of performing the heat treatment. Specifically, the endoscope system 1 illustrated in FIG. 1 is used for the transurethral resection of the bladder tumor (TUR-Bt), and is used when performing the treatment on a tumor (bladder cancer) of the bladder or the pathogenic region.

The endoscope system 1 illustrated in FIG. 1 includes an insertion unit 2, a light source device 3, a light guide 4, an endoscope camera head 5 (endoscope imaging device), a first transmission cable 6, a display device 7, a second transmission cable 8, a control device 9, a third transmission cable 10, a perfusion device 11, and a fourth transmission cable 12.

The insertion unit 2 is rigid or at least partially flexible and has an elongated shape. The insertion unit 2 is inserted into the subject such as a patient via a trocar. The insertion unit 2 is provided with an optical system such as a lens that forms the observation image therein.

The light source device 3 is connected to one end of the light guide 4 and supplies illumination light for irradiating the inside of the subject to one end of the light guide 4 under the control of the control device 9. The light source device 3 is implemented by using one or more light sources of any one of semiconductor laser elements such as a light emitting diode (LED) light source, a xenon lamp, and a laser diode (LD), a processor that is a processing device including hardware such as a field programmable gate array (FPGA) and a central processing unit (CPU), and a memory that is a temporary storage area used by the processor. The light source device 3 and the control device 9 may be configured to perform communication individually as illustrated in FIG. 1, or may be integrated with each other.

The light guide 4 has one end detachably connected to the light source device 3, and the other end detachably connected to the insertion unit 2. The light guide 4 guides the illumination light supplied from the light source device 3 from one end to the other end and supplies the illumination light to the insertion unit 2.

An eyepiece portion 21 of the insertion unit 2 is detachably connected to the endoscope camera head 5. The endoscope camera head 5 generates the imaging signal (RAW data) by receiving the observation image formed by the insertion unit 2 and performing photoelectric conversion, and outputs the imaging signal to the control device 9 via the first transmission cable 6 under the control of the control device 9.

The first transmission cable 6 has one end detachably connected to the control device 9 via a video connector 61, and the other end detachably connected to the endoscope camera head 5 via a camera head connector 62. The first transmission cable 6 transmits the imaging signal output from the endoscope camera head 5 to the control device 9, and transmits setting data, power, and the like output from the control device 9 to the endoscope camera head 5. Here, the setting data is a control signal, a synchronization signal, a clock signal, and the like for controlling the endoscope camera head 5.

The display device 7 displays the observation image based on the imaging signal subjected to image processing in the control device 9 and various types of information regarding the endoscope system 1 under the control of the control device 9. The display device 7 is implemented by using a display monitor such as liquid crystal or organic electro luminescence (EL).

The second transmission cable 8 has one end detachably connected to the display device 7, and the other end detachably connected to the control device 9. The second transmission cable 8 transmits the imaging signal subjected to the image processing in the control device 9 to the display device 7.

The control device 9 is implemented by using a processor that is a processing device including hardware such as a graphics processing unit (GPU), an FPGA, or a CPU, and a memory that is a temporary storage area used by the processor. The control device 9 integrally controls operations of the light source device 3, the endoscope camera head 5, and the display device 7 via each of the first transmission cable 6, the second transmission cable 8, and the third transmission cable 10 according to a program recorded in the memory. In addition, the control device 9 performs various types of image processing on the imaging signal input via the first transmission cable 6 and outputs the imaging signal to the second transmission cable 8.

The third transmission cable 10 has one end detachably connected to the light source device 3, and the other end detachably connected to the control device 9. The third transmission cable 10 transmits control data from the control device 9 to the light source device 3.

The perfusion device 11 supplies a perfusate such as sterilized physiological saline into the bladder of the subject from a liquid feeding hole (not illustrated) of the insertion unit 2 via a liquid feeding tube (not illustrated) under the control of the control device 9. The perfusion device 11 is implemented using a liquid feeding pump, a waste liquid pump, a storage tank for storing the perfusate, a waste liquid tank for storing a discharged perfusate, or the like.

The fourth transmission cable 12 has one end detachably connected to the perfusion device 11, and the other end detachably connected to the control device 9. The fourth transmission cable 12 transmits the control data from the control device 9 to the perfusion device 11.

### Functional Configuration of Main Part of Endoscope System

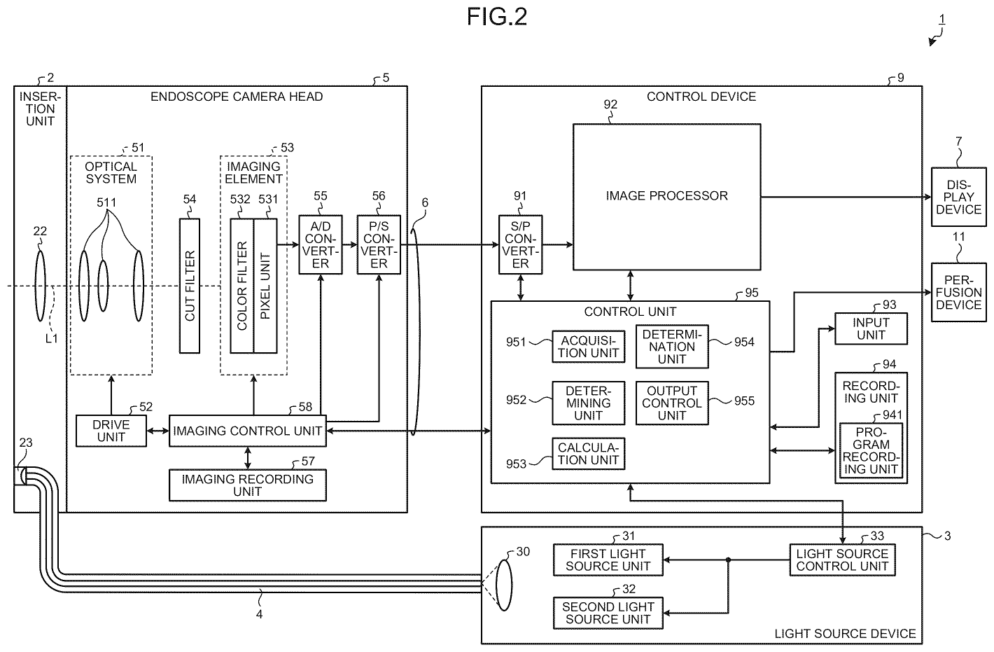

Next, a functional configuration of a main part of the above-described endoscope system 1 will be described. FIG. 2 is a block diagram illustrating the functional configuration of the main part of the endoscope system 1.

### Configuration of Insertion Unit

First, a configuration of the insertion unit 2 will be described. The insertion unit 2 includes an optical system 22 and an illumination optical system 23.

The optical system 22 forms a subject image by collecting light such as reflected light reflected from the subject, return light from the subject, excitation light from the subject, and fluorescence emitted from a thermally denatured region thermally denatured by the heat treatment using the energy device or the like. The optical system 22 is implemented by using one or more lenses or the like.

The illumination optical system 23 irradiates the subject with the illumination light supplied from the light guide 4. The illumination optical system 23 is implemented by using one or more lenses or the like.

### Configuration of Light Source Device

Next, a configuration of the light source device 3 will be described. The light source device 3 includes a condenser lens 30, a first light source unit 31, a second light source unit 32, and a light source control unit 33.

The condenser lens 30 condenses light emitted by each of the first light source unit 31 and the second light source unit 32 and emits the light to the light guide 4. The first light source unit 31 supplies white light as illumination light to the light guide 4 by emitting the white light (normal light) that is visible light under the control of the light source control unit 33. The first light source unit 31 is implemented using a collimator lens, a white LED lamp, a driver, or the like. The first light source unit 31 may supply the white light that is the visible light by simultaneously performing light emission using a red LED lamp, a green LED lamp, and a blue LED lamp. It is a matter of course that the first light source unit 31 may be implemented using a halogen lamp, a xenon lamp, or the like.

The second light source unit 32 emits the excitation light having a predetermined wavelength band to supply the excitation light to the light guide 4 as the illumination light under the control of the light source control unit 33. Here, the excitation light has a wavelength band of 400 nm to 430 nm (a central wavelength is 415 nm). The second light source unit 32 is implemented by using a semiconductor laser such as a collimator lens or a violet laser diode (LD), a drive driver, and the like. In the first embodiment, the excitation light excites advanced glycation end products generated by performing the heat treatment on the biological tissue using the energy device or the like. In a case where an amino acid and a reducing sugar are heated, a saccharification reaction (Maillard reaction) occurs. The end products resulting from the Maillard reaction are generally called the advanced glycation end products (AGEs). As a characteristic of the AGEs, it is known that a substance having a fluorescence characteristic is contained. That is, in a case where the biological tissue is subjected to the heat treatment by the energy device, the AGEs are generated when the Maillard reaction occurs by heating the amino acid and the reducing sugar in the biological tissue. The AGEs generated by the heating can visualize a state of the heat treatment by fluorescence observation. Furthermore, the AGEs are known to emit stronger fluorescence than an autofluorescent substance originally present in the biological tissue. That is, in the first embodiment, the thermally denatured region obtained by the heat treatment is visualized using the fluorescence characteristic of the AGEs generated in the biological tissue by the heat treatment using the energy device or the like. Therefore, in the first embodiment, the biological tissue is irradiated with the excitation light of blue light having a wavelength of about 415 nm for exciting the AGEs from the second light source unit 32. As a result, in the first embodiment, a fluorescence image (thermal denaturation image) can be observed based on the imaging signal obtained by imaging the fluorescence (for example, green light having a wavelength of 490 to 625 nm) emitted from the thermally denatured region generated from the AGEs.

The light source control unit 33 is implemented by using a processor including hardware such as an FPGA or a CPU, and a memory that is a temporary storage area used by the processor. The light source control unit 33 controls a light emission timing, a light emission time, and the like of each of the first light source unit 31 and the second light source unit 32 based on the control data input from the control device 9.

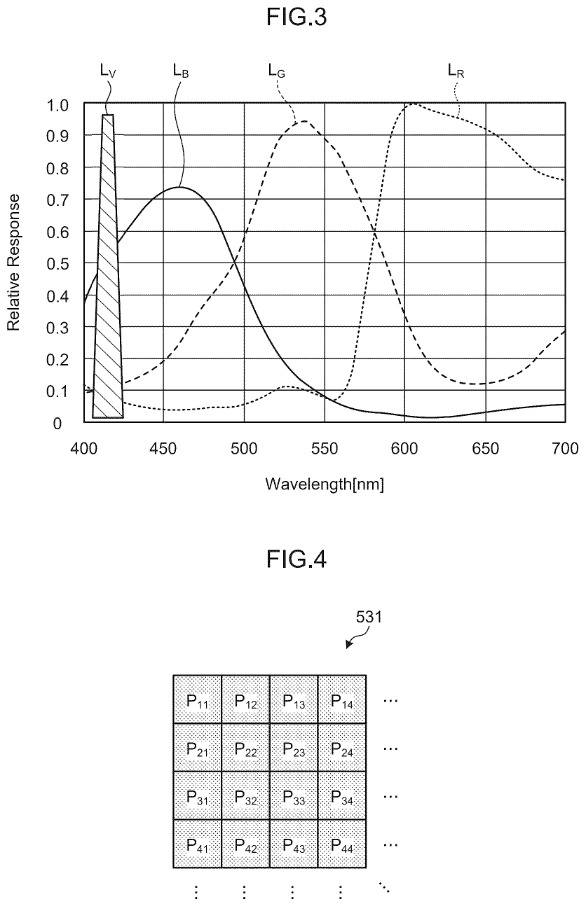

Here, a wavelength characteristic of the light emitted by the second light source unit 32 will be described. FIG. 3 is a diagram schematically illustrating the wavelength characteristic of the excitation light emitted by the second light source unit 32. In FIG. 3, a horizontal axis represents the wavelength (nm), and a vertical axis represents the wavelength characteristic. In FIG. 3, a polygonal line L_{V} indicates the wavelength characteristic of the excitation light emitted by the second light source unit 32. In FIG. 3, a curve L_{B} indicates a blue wavelength band, a curve L_{G} indicates a green wavelength band, and a curve L_{R} indicates a red wavelength band.

As indicated by the polygonal line L_{V} in FIG. 3, the second light source unit 32 emits the excitation light having the central wavelength (peak wavelength) of 415 nm and the wavelength band of 400 nm to 430 nm.

### Configuration of Endoscope Camera Head

Returning to FIG. 2, the description of the configuration of the endoscope system 1 will be continued.

Next, a configuration of the endoscope camera head 5 will be described. The endoscope camera head 5 includes an optical system 51, a drive unit 52, an imaging element 53, a cut filter 54, an A/D converter 55, a P/S converter 56, an imaging recording unit 57, and an imaging control unit 58.

The optical system 51 forms the subject image condensed by the optical system 22 of the insertion unit 2 on a light receiving surface of the imaging element 53. The optical system 51 can change a focal length and a focal position. The optical system 51 is implemented using a plurality of lenses 511. The optical system 51 changes the focal length and the focal position by moving each of the plurality of lenses 511 on an optical axis L1 by the drive unit 52.

The drive unit 52 moves the plurality of lenses 511 of the optical system 51 along the optical axis L1 under the control of the imaging control unit 58. The drive unit 52 is implemented using a motor such as a stepping motor, a DC motor, or a voice coil motor, and a transmission mechanism such as a gear that transmits rotation of the motor to the optical system 51.

The imaging element 53 is implemented by using a charge coupled device (CCD) or complementary metal oxide semiconductor (CMOS) image sensor including a plurality of pixels arranged in a two-dimensional matrix. The imaging element 53 receives the subject image (light beam) formed by the optical system 51 and passing through the cut filter 54, performs the photoelectric conversion to generate the imaging signal (RAW data), and outputs the imaging signal to the A/D converter 55 under the control of the imaging control unit 58. The imaging element 53 includes a pixel unit 531 and a color filter 532.

FIG. 4 is a diagram schematically illustrating a configuration of the pixel unit 531. As illustrated in FIG. 4, in the pixel unit 531, a plurality of pixels Pₙₘ (n=an integer of 1 or more, and m=an integer of 1 or more) such as photodiodes that accumulate charges according to a light quantity are arranged in a two-dimensional matrix. The pixel unit 531 reads an image signal as the image data from a pixel Pₙₘ in a reading region arbitrarily set as a reading target among the plurality of pixels Pₙₘ, and outputs the image signal to the A/D converter 55 under the control of the imaging control unit 58.

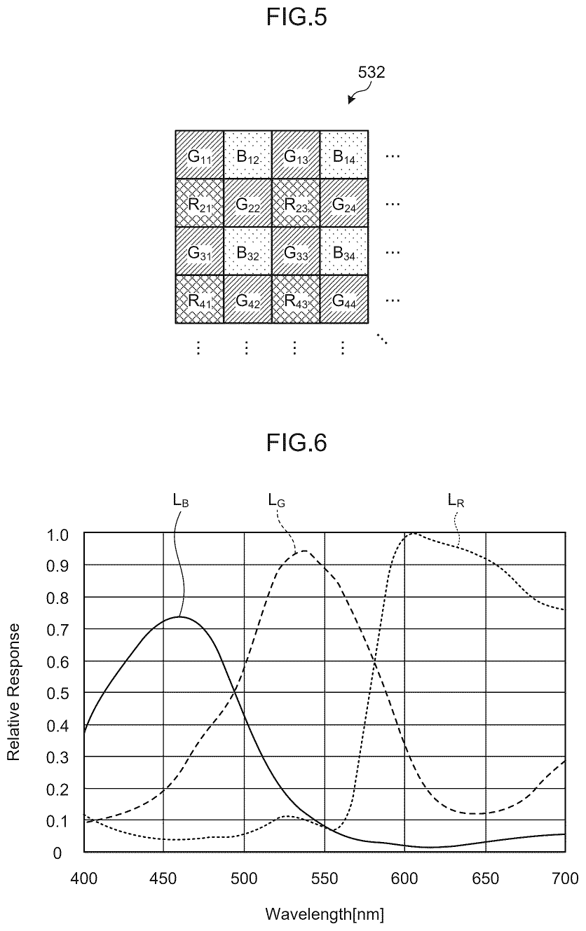

FIG. 5 is a diagram schematically illustrating a configuration of the color filter 532. As illustrated in FIG. 5, the color filter 532 is implemented by a Bayer array having 2×2 as one unit. The color filter 532 is implemented using a filter R that transmits light in the red wavelength band, two filters G that transmit light in the green wavelength band, and a filter B that transmits light in the blue wavelength band.

FIG. 6 is a diagram schematically illustrating sensitivity and a wavelength band of each filter. In FIG. 6, a horizontal axis represents the wavelength (nm), and a vertical axis represents a transmission characteristic (sensitivity characteristic). In FIG. 6, the curve L_{B} indicates the transmission characteristic of the filter B, the curve L_{G} indicates the transmission characteristic of the filter G, and the curve L_{R} indicates the transmission characteristic of the filter R.

As indicated by the curve L_{B} in FIG. 6, the filter B transmits light in the blue wavelength band. As indicated by the curve L_{G} in FIG. 6, the filter G transmits light in the green wavelength band. Further, as indicated by the curve L_{R} in FIG. 6, the filter R transmits light in the red wavelength band. In the following description, a pixel Pₙₘ in which the filter R is disposed on a light receiving surface will be described as an R pixel, a pixel Pₙₘ in which the filter G is disposed on a light receiving surface will be described as a G pixel, and a pixel Pₙₘ in which the filter B is disposed on a light receiving surface will be described as a B pixel.

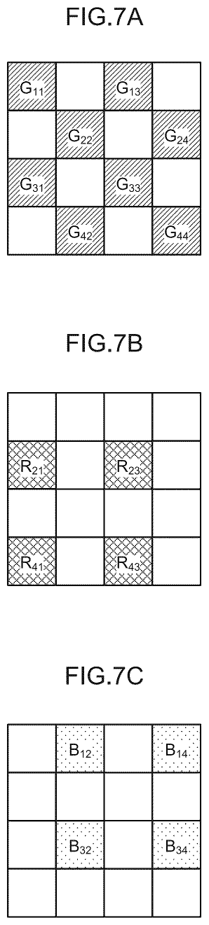

With the imaging element 53 configured as described above, in a case where the subject image formed by the optical system 51 is received, as illustrated in FIGS. 7A to 7C, color signals (an R signal, a G signal, and a B signal) of the R pixel, the G pixel, and the B pixel are generated.

Returning to FIG. 2, the description of the configuration of the endoscope system 1 will be continued.

The cut filter 54 is disposed on the optical axis L1 between the optical system 51 and the imaging element 53. The cut filter 54 is provided on a light receiving surface side (incident surface side) of the G pixel provided with the filter G that transmits at least the green wavelength band of the color filter 532. The cut filter 54 blocks light in a shorter wavelength band including the wavelength band of the excitation light, and transmits a longer wavelength band beyond the wavelength band of the excitation light.

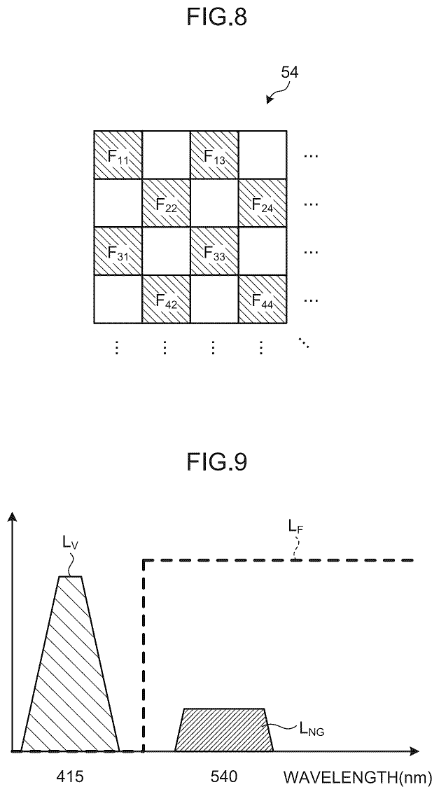

FIG. 8 is a diagram schematically illustrating a configuration of the cut filter 54. As illustrated in FIG. 8, a filter F₁₁ included in the cut filter 54 is disposed at a position where a filter G₁₁ (see FIG. 5) is disposed, on the light receiving surface side immediately above the filter G₁₁.

FIG. 9 is a diagram schematically illustrating a transmission characteristic of the cut filter 54. In FIG. 9, a horizontal axis represents the wavelength (nm), and a vertical axis represents the transmission characteristic. In FIG. 9, a polygonal line L_{F} indicates the transmission characteristic of the cut filter 54, a polygonal line LNG indicates a wavelength characteristic of the fluorescence, and the polygonal line L_{V} indicates the wavelength characteristic of the excitation light.

As illustrated in FIG. 9, the cut filter 54 blocks the wavelength band of the excitation light and transmits a longer wavelength band beyond the wavelength band of the excitation light. Specifically, the cut filter 54 blocks light in a shorter wavelength band of 400 nm to less than 430 nm including the wavelength band of the excitation light, and transmits light in a longer wavelength band beyond the wavelength band of 400 nm to 430 nm including the excitation light.

Returning to FIG. 2, the description of the configuration of the endoscope camera head 5 will be continued.

The A/D converter 55 executes A/D conversion processing on the analog imaging signal input from the imaging element 53, and outputs the analog imaging signal to the P/S converter 56 under the control of the imaging control unit 58. The A/D converter 55 is implemented by using an A/D conversion circuit or the like.

The P/S converter 56 performs parallel/serial conversion on the digital imaging signal input from the A/D converter 55, and outputs the imaging signal subjected to the parallel/serial conversion to the control device 9 via the first transmission cable 6 under the control of the imaging control unit 58. The P/S converter 56 is implemented by using a P/S conversion circuit or the like. In the first embodiment, an E/O converter that converts the imaging signal into an optical signal may be provided instead of the P/S converter 56, and the imaging signal may be output to the control device 9 by the optical signal, or the imaging signal may be transmitted to the control device 9 by wireless communication such as Wireless Fidelity (Wi-Fi) (registered trademark).

The imaging recording unit 57 records various types of information (for example, pixel information of the imaging element 53 and the characteristic of the cut filter 54) regarding the endoscope camera head 5. Furthermore, the imaging recording unit 57 records various types of setting data and control parameters transmitted from the control device 9 via the first transmission cable 6. The imaging recording unit 57 is implemented using a nonvolatile memory or a volatile memory.

The imaging control unit 58 controls an operation of each of the drive unit 52, the imaging element 53, the A/D converter 55, and the P/S converter 56 based on the setting data received from the control device 9 via the first transmission cable 6. The imaging control unit 58 is implemented by using a timing generator (TG), a processor including hardware such as an application specific integrated circuit (ASIC) or a CPU, and a memory that is a temporary storage area used by the processor.

### Configuration of Control Device

Next, a configuration of the control device 9 will be described.

The control device 9 includes an S/P converter 91, an image processor 92, an input unit 93, a recording unit 94, and a control unit 95.

The S/P converter 91 performs serial/parallel conversion on the image data received from the endoscope camera head 5 via the first transmission cable 6 and outputs the image data to the image processor 92 under the control of the control unit 95. In a case where the endoscope camera head 5 outputs the imaging signal as the optical signal, an O/E converter that converts the optical signal into an electric signal may be provided instead of the S/P converter 91. Furthermore, in a case where the endoscope camera head 5 transmits the imaging signal by wireless communication, a communication module capable of receiving a wireless signal may be provided instead of the S/P converter 91.

The image processor 92 executes predetermined image processing on the imaging signal of the parallel data input from the S/P converter 91 and outputs the imaging signal to the display device 7 under the control of the control unit 95. Here, the predetermined image processing is demosaic processing, white balance processing, gain adjustment processing, y correction processing, format conversion processing, or the like. The image processor 92 is implemented by using a processor that is a processing device including hardware such as a GPU or an FPGA and a memory that is a temporary storage area used by the processor.

The input unit 93 receives various operations related to the endoscope system 1 and outputs the received operations to the control unit 95. The input unit 93 is implemented using a mouse, a foot switch, a keyboard, a button, a switch, a touch panel, or the like.

The recording unit 94 is implemented by using a recording medium such as a volatile memory, a nonvolatile memory, a solid state drive (SSD), a hard disk drive (HDD), or a memory card. The recording unit 94 records data including various parameters and the like necessary for an operation of the endoscope system 1. Furthermore, the recording unit 94 includes a program recording unit 941 that records various programs for operating the endoscope system 1.

The control unit 95 is implemented by using a processor including hardware such as an FPGA or a CPU, and a memory that is a temporary storage area used by the processor. The control unit 95 integrally controls each unit included in the endoscope system 1. Specifically, the control unit 95 reads and executes the program recorded in the program recording unit 941 in a work area of the memory, and controls each component and the like through the execution of the program by the processor, so that the hardware and software cooperate with each other to implement a functional module matching a predetermined purpose. Specifically, the control unit 95 includes an acquisition unit 951, a determining unit 952, a calculation unit 953, a determination unit 954, and an output control unit 955.

The acquisition unit 951 acquires the imaging signal generated by imaging performed by the endoscope camera head 5 via the insertion unit 2.

The determining unit 952 determines first information included in a first fluorescence image. Specifically, the determining unit 952 determines a fluorescence amount of the first fluorescence image as the first information based on a pixel value included in the first fluorescence image. In addition, the determining unit 952 determines second information included in a second fluorescence image. Specifically, the determining unit 952 determines a fluorescence amount of the second fluorescence image as the second information based on the pixel value included in the second fluorescence image.

The calculation unit 953 calculates a difference between the fluorescence amount of the first fluorescence image determined by the determining unit 952 and the fluorescence amount of the second fluorescence image determined by the determining unit 952. Specifically, the calculation unit 953 calculates a temporal change amount of the fluorescence amount as the difference based on the fluorescence amount of the first fluorescence image determined by the determining unit 952 and the fluorescence amount of the second fluorescence image determined by the determining unit 952.

The determination unit 954 determines whether or not the difference between the fluorescence amount of the first fluorescence image and the fluorescence amount of the second fluorescence image calculated by the calculation unit 953 is less than a predetermined value. Specifically, the determination unit 954 determines whether or not the change amount of the fluorescence amount is less than the predetermined value as the difference between the fluorescence amounts calculated by the calculation unit 953.

The output control unit 955 outputs, to the display device 7, a drive signal for causing the display device 7 to display information indicating that discharge of a resected piece has been completed as discharge information indicating a discharge status of the resected piece resected by a resection treatment tool in a target region. In addition, the output control unit 955 outputs, to the display device 7, a drive signal for causing the display device 7 to display information indicating that the resected piece is being discharged as the discharge information indicating the discharge status of the resected piece resected by the resection treatment tool in the target region.

### Processing in Control Device

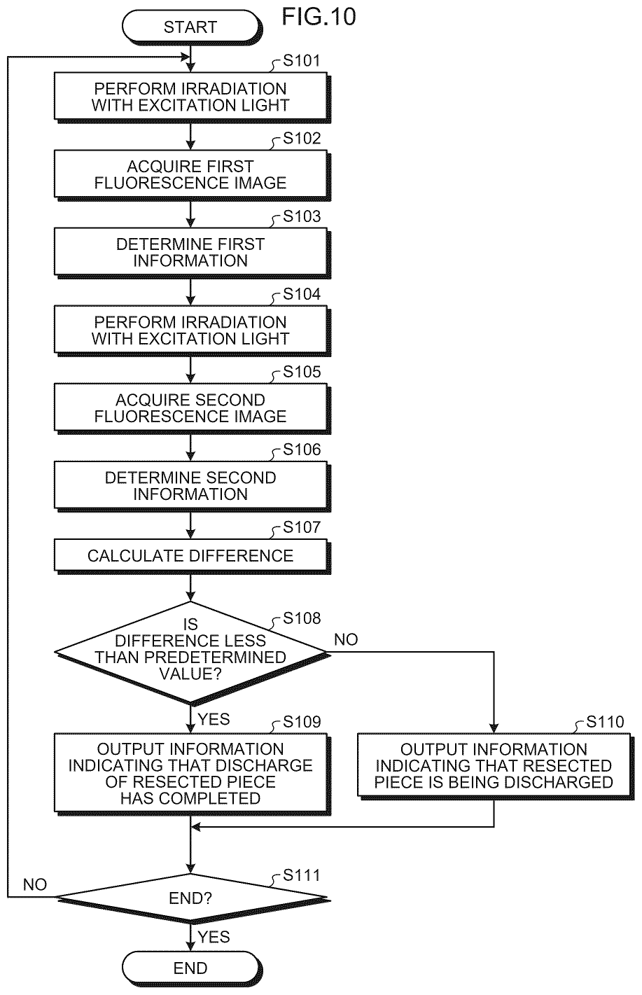

Next, processing executed by the control device 9 will be described. FIG. 10 is a flowchart illustrating an outline of processing executed by the control device 9.

As illustrated in FIG. 10, first, the control unit 95 causes the second light source unit 32 of the light source device 3 to emit the light to supply the excitation light to the insertion unit 2, thereby irradiating the target region of the biological tissue with the excitation light (Step S101).

Subsequently, the acquisition unit 951 acquires the first fluorescence image generated by imaging performed by the endoscope camera head 5 via the insertion unit 2 (Step S102).

Thereafter, the determining unit 952 determines the first information included in the first fluorescence image (Step S103). Specifically, the determining unit 952 determines the fluorescence amount of the first fluorescence image as the first information based on the pixel value included in the first fluorescence image.

Subsequently, the control unit 95 causes the second light source unit 32 of the light source device 3 to emit light to supply the excitation light to the insertion unit 2, thereby irradiating the target region of the biological tissue with the excitation light (Step S104).

Thereafter, the acquisition unit 951 acquires the second fluorescence image generated by imaging the target region after a time at which the first fluorescence image is captured by the endoscope camera head 5 via the insertion unit 2 (Step S105).

Subsequently, the determining unit 952 determines the second information included in the second fluorescence image (Step S106). Specifically, the determining unit 952 determines a value obtained by averaging the pixel values of the first fluorescence image and the second fluorescence image as the fluorescence amount.

The calculation unit 953 calculates the difference between the fluorescence amount of the first fluorescence image determined by the determining unit 952 and the fluorescence amount of the second fluorescence image determined by the determining unit 952 (Step S107). Specifically, the calculation unit 953 calculates a temporal change amount of the fluorescence amount as the difference based on the fluorescence amount of the first fluorescence image determined by the determining unit 952 and the fluorescence amount of the second fluorescence image determined by the determining unit 952. The calculation unit 953 may divide each of the first fluorescence image and the second fluorescence image into a plurality of regions, calculate an average value of the pixel values (fluorescence amounts) of the pixels included in each of the plurality of regions, and then calculate the temporal change amount of the fluorescence amount using the average value of the same region of each of the first fluorescence image and the second fluorescence image. Furthermore, the calculation unit 953 may calculate the temporal change amount of the fluorescence amount from a distribution of the fluorescence amount of each of the first fluorescence image and the second fluorescence image.

The determination unit 954 determines whether or not the change amount of the fluorescence amount calculated by the calculation unit 953 is less than the predetermined value (Step S108). In a case where the determination unit 954 determines that the change amount of the fluorescence amount calculated by the calculation unit 953 is less than the predetermined value (Step S108: Yes), the control device 9 proceeds to Step S109 described below. On the other hand, in a case where the determination unit 954 determines that the change amount of the fluorescence amount calculated by the calculation unit 953 is not less than the predetermined value (Step S108: No), the control device 9 proceeds to Step S110 described below.

In Step S109, the output control unit 955 outputs, to the display device 7, the drive signal for causing the display device 7 to display the information indicating that the discharge of the resected piece has been completed as the discharge information indicating the discharge status of the resected piece resected by the resection treatment tool in the target region. In this case, the display device 7 superimposes and displays the information indicating that the discharge of the resected piece has been completed, for example, characters, figures, symbols, and the like such as “discharge completed” on a display image input from the image processor 92 according to the drive signal input from the control unit 95. As a result, a user can grasp that the resected piece is excluded from the target region. After Step S109, the control device 9 proceeds to Step S111 described below.

In Step S110, the output control unit 955 outputs, to the display device 7, the drive signal for causing the display device 7 to display the information indicating that the resected piece is being discharged as the discharge information indicating the discharge status of the resected piece resected by the resection treatment tool in the target region. In this case, the display device 7 superimposes and displays characters, figures, symbols, and the like indicating that the resected piece is being discharged, for example, “being discharged” and the like on the display image input from the image processor 92 according to the drive signal input from the control unit 95. As a result, the user can grasp that the resected piece is excluded from the target region. After Step S109, the control device 9 proceeds to Step S111 described below.

In Step S111, the determination unit 954 determines whether or not an end signal for ending the observation of the subject by the endoscope system 1 has been input from the input unit 93. In a case where the determination unit 954 determines that the end signal for ending the observation of the subject by the endoscope system 1 has been input from the input unit 93 (Step S111: Yes), the control device 9 ends the processing. On the other hand, in a case where the determination unit 954 determines that the end signal for ending the observation of the subject by the endoscope system 1 has not been input from the input unit 93 (Step S111: No), the control device 9 returns to Step S101 described above.

According to the first embodiment described above, the output control unit 955 outputs, to the display device 7, the drive signal for causing the display device 7 to display the information indicating that the discharge of the resected piece has been completed as the discharge information indicating the discharge status of the resected piece resected by the resection treatment tool in the target region, so that it is possible to grasp the status of the resected piece in the perfusate supplied into the organ.

In addition, according to the first embodiment, the output control unit 955 outputs, to the display device 7, the drive signal for causing the display device 7 to display the information indicating that the resected piece is being discharged as the discharge information indicating the discharge status of the resected piece resected by the resection treatment tool in the target region, so that it is possible to grasp the status of the resected piece in the perfusate.

The calculation unit 953 calculates, as the difference, the change amount between the fluorescence amount of the first fluorescence image determined by the determining unit 952 and the fluorescence amount of the second fluorescence image determined by the determining unit 952, and may calculate a motion vector based on the first fluorescence image and the second fluorescence image. In this case, the calculation unit 953 calculates the motion vector based on difference information between the first fluorescence image and the second fluorescence image. The calculation unit 953 calculates the motion vector by referring to a known technology of calculating (estimating) an optical flow, for example, the Lucas-Kanade method or the Horm-Schunk method. At this time, the determination unit 954 determines the subject having a large motion vector calculated by the calculation unit 953 as the resected piece, and determines whether or not the motion vector is less than a predetermined value in the target region. That is, the determination unit 954 determines that the resected piece has been discharged from the target region in a case where the motion vector is less than the predetermined value, and determines that the resected piece is being discharged from the inside of the target region in a case where the motion vector is not less than the predetermined value.

### Second Embodiment

Next, a second embodiment will be described. An endoscope system according to the second embodiment has the same configuration as the endoscope system 1 according to the first embodiment described above, and processing executed by a control device 9 is different. Specifically, in the first embodiment described above, the drive signal is output to the display device 7, but in the second embodiment, a drive signal is output to a perfusion device 11. Therefore, processing executed by a control device included in an endoscope system according to the second embodiment will be described below.

### Processing in Control Device

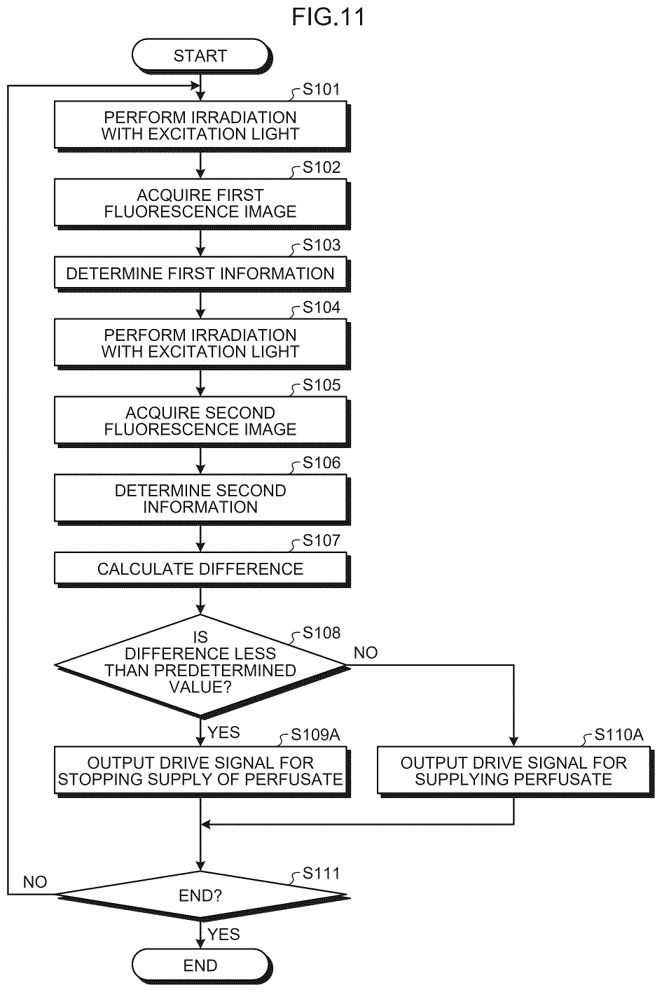

FIG. 11 is a flowchart illustrating an outline of processing executed by a control device 9 according to the second embodiment. In FIG. 11, the control device 9 executes Step S109A and Step S110A instead of Step S109 and Step S110 of FIG. 10 described above, and Steps S109A and S110A will be described since the other steps are similar to the steps described above.

In Step S109A, an output control unit 955 outputs the drive signal for stopping supply of a perfusate to the perfusion device 11 as a signal for controlling the perfusion device 11 that supplies the perfusate toward a target region. As a result, a user does not determine a state of the perfusate by an empirical rule, it is possible to concentrate on a medical procedure of a subject. After Step S109A, the control device 9 proceeds to Step S111.

In Step S110A, the output control unit 955 outputs the drive signal for supplying the perfusate to the perfusion device 11 as a signal for controlling the perfusion device 11 that supplies the perfusate toward the target region. As a result, the perfusion device 11 supplies the perfusate toward the target region, so that an operator can discharge a resected piece resected by a resection treatment tool to the outside. After Step S110A, the control device 9 proceeds to Step S111.

According to the second embodiment described above, it is possible to obtain an effect similar to that of the first embodiment described above, that is, it is possible to grasp a status of the resected piece in the perfusate.

### Third Embodiment

Next, a third embodiment will be described. In the first embodiment described above, the control unit 95 of the control device 9 outputs the drive signal to the display device 7, but in the third embodiment, a medical device that outputs a drive signal for driving a display device 7 is separately provided. Hereinafter, a configuration of an endoscope system according to the third embodiment will be described. Note that the same components as those of the endoscope system 1 according to the first embodiment described above are denoted by the same reference signs, and a detailed description thereof will be omitted.

### Configuration of Endoscope System

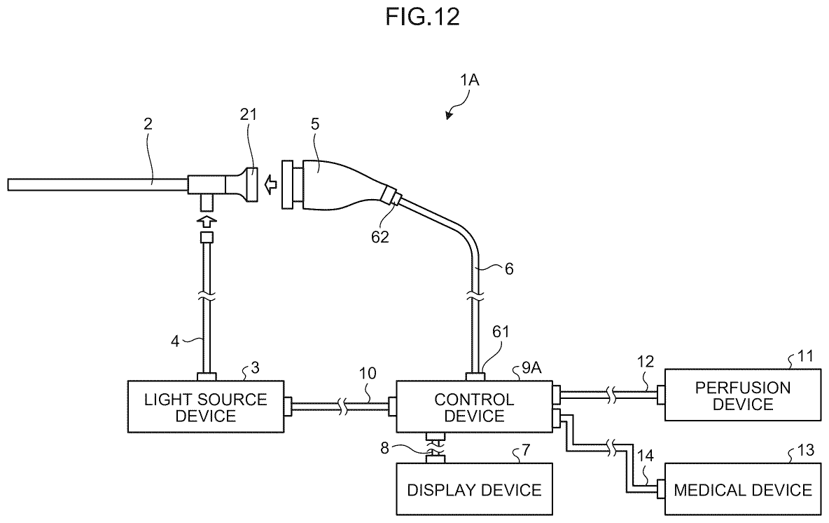

FIG. 12 is a diagram illustrating a configuration of the endoscope system according to the third embodiment. An endoscope system 1A illustrated in FIG. 12 includes a control device 9A instead of the control device 9 of the endoscope system 1 according to the first embodiment described above. Furthermore, the endoscope system 1A further includes a medical device 13 and a fifth transmission cable 14 in addition to the configuration of the endoscope system 1 according to the first embodiment described above.

The control device 9A is implemented by using a processor that is a processing device including hardware such as a GPU, an FPGA, or a CPU, and a memory that is a temporary storage area used by the processor. The control device 9A integrally controls operations of a light source device 3, an endoscope camera head 5, the display device 7, and the medical device 13 via each of a first transmission cable 6, a second transmission cable 8, a third transmission cable 10, and a fourth transmission cable 12 according to a program recorded in the memory. The control device 9A is different from the control unit 95 according to the above-described first embodiment in that the functions of an acquisition unit 951, a determining unit 952, a calculation unit 953, a determination unit 954, and an output control unit 955 are not provided.

The medical device 13 is implemented by using a processor that is a processing device including hardware such as a GPU, an FPGA, or a CPU, and a memory that is a temporary storage area used by the processor. The medical device 13 acquires various types of information from the control device 9A via the fifth transmission cable 14, and outputs the acquired various types of information to the control device 9A. Note that a detailed functional configuration of the medical device 13 will be described below.

The fifth transmission cable 14 has one end detachably connected to the control device 9A, and the other end detachably connected to the medical device 13. The fifth transmission cable 14 transmits various types of information from the control device 9A to the medical device 13, and transmits various types of information from the medical device 13 to the control device 9A.

### Functional Configuration of Medical Device

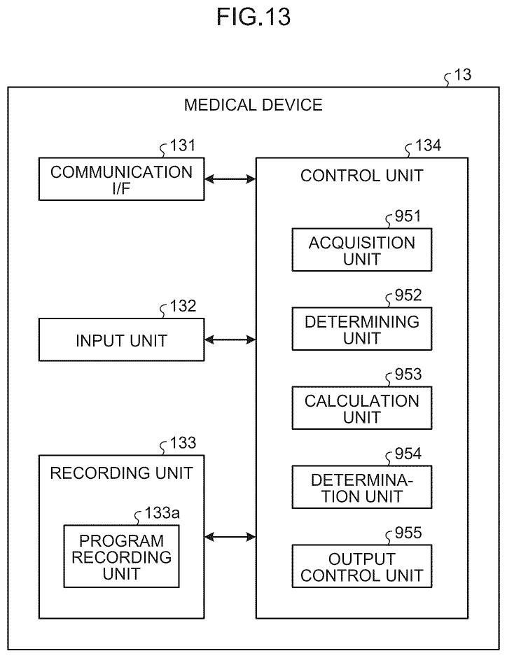

FIG. 13 is a block diagram illustrating a functional configuration of the medical device 13. The medical device 13 illustrated in FIG. 16 includes a communication I/F 131, an input unit 132, a recording unit 133, and a control unit 134.

The communication I/F 131 is an interface for communicating with the control device 9A via the fifth transmission cable 14. The communication I/F 131 receives various types of information from the control device 9A according to a predetermined communication standard, and outputs the received various types of information to the control unit 134.

The input unit 132 receives inputs of various operations related to the endoscope system 1A and outputs the received operations to the control unit 134. The input unit 132 is implemented using a mouse, a foot switch, a keyboard, a button, a switch, a touch panel, and the like.

The recording unit 133 is implemented by using a recording medium such as a volatile memory, a nonvolatile memory, an SSD, an HDD, or a memory card. The recording unit 133 records data including various parameters and the like necessary for an operation of the medical device 13. Furthermore, the recording unit 133 includes a program recording unit 133a that records various programs for operating the medical device 13.

The control unit 134 is implemented by using a processor including hardware such as an FPGA or a CPU, and a memory that is a temporary storage area used by the processor. The control unit 134 integrally controls each unit included in the medical device 13. The control unit 134 has the same function as the control unit 95 according to the first embodiment described above. Specifically, the control unit 134 includes the acquisition unit 951, the determining unit 952, the calculation unit 953, the determination unit 954, and the output control unit 955.

The medical device 13 configured as described above executes processing similar to that of the control device 9 according to the first embodiment described above, and outputs a processing result to the control device 9A. In this case, the control device 9A causes an image processor 92 to output a display image according to the presence or absence of a light emission region in a detection range R1 of a white image generated by the image processor 92 based on the processing result of the medical device 13, and causes the display device 7 to display the display image.

According to the second embodiment described above, it is possible to obtain an effect similar to that of the first embodiment described above, and the user can confirm a state of thermal denaturation in a specific region.

### OTHER EMBODIMENTS

Various embodiments can be formed by appropriately combining a plurality of constituent elements disclosed in the endoscope systems according to the first to third embodiments of the present disclosure described above. For example, some constituent elements may be deleted from all the constituent elements described in the endoscope systems according to the embodiments of the present disclosure described above. Furthermore, the constituent elements described in the endoscope systems according to the embodiments of the present disclosure described above may be appropriately combined.

Furthermore, in the endoscope systems according to the first to third embodiments of the present disclosure, the constituent elements are connected to each other in a wired manner, or may be connected wirelessly via a network.

Furthermore, in the first to third embodiments of the present disclosure, the function of the control unit included in the endoscope system, and the functional modules of the acquisition unit 951, the determining unit 952, the calculation unit 953, the determination unit 954, and the output control unit 955 may be provided in a server or the like connectable via a network. It is a matter of course that a server may be provided for each functional module.

In addition, in the first to third embodiments of the present disclosure, an example in which the endoscope system is used for the transurethral resection of the bladder tumor has been described, but the present disclosure is not limited thereto, and the endoscope system can be applied to various medical procedures for resection of a lesion by, for example, the energy device or the like.

Furthermore, in the endoscope systems according to the first to third embodiments of the present disclosure, the “unit” described above can be replaced with “means”, “circuit”, or the like. For example, the control unit can be replaced with control means or a control circuit.

Note that, in the description of the flowcharts in the present specification, the context of processing between steps is clearly indicated using expressions such as “first”, “thereafter”, and “subsequently”, but the order of processing necessary for implementing the embodiments is not uniquely determined by these expressions. That is, the order of processing in the flowcharts described in the present specification can be changed within a range without inconsistency.

According to the present disclosure, it is possible to grasp a status of a resected piece in a perfusate.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical device comprising a processor comprising hardware, the processor being configured to
acquire a first fluorescence image obtained by imaging a target region and a second fluorescence image obtained by imaging the target region after a time at which the first fluorescence image is captured,
generate a drive signal for causing a display device to display discharge information indicating a discharge status of a resected piece resected by a resection treatment tool in the target region based on first information included in the first fluorescence image and second information included in the second fluorescence image, and
output the drive signal.

2. The medical device according to claim 1, wherein
the drive signal includes a control signal for controlling a perfusion device configured to supply a perfusate toward the target region.

3. The medical device according to claim 1, wherein
each of the first information and the second information is a fluorescence amount, and
the processor is further configured to
calculate a difference between the fluorescence amount of the first fluorescence image and the fluorescence amount of the second fluorescence image,
determine whether or not the difference is less than a predetermined value, and
output the drive signal for causing the display device to display information indicating that discharge of the resected piece has been completed as the discharge information when it is determined that the difference is less than the predetermined value.

4. The medical device according to claim 3, wherein
the processor is further configured to output the drive signal for causing the display device to display information indicating that the resected piece is being discharged as the discharge information when it is determined that the difference is not less than the predetermined value.

5. The medical device according to claim 2, wherein
each of the first information and the second information is a fluorescence amount, and
the processor is further configured to
calculate a difference between the fluorescence amount of the first fluorescence image and the fluorescence amount of the second fluorescence image,
determine whether or not the difference is less than a predetermined value, and
output the drive signal for stopping the supply of the perfusate to the perfusion device as the control signal when it is determined that the difference is less than the predetermined value.

6. The medical device according to claim 5, wherein
the processor is further configured to output the drive signal for supplying the perfusate to the perfusion device as the control signal when it is determined that the difference is not less than the predetermined value.

7. A medical system comprising:
a light source device including a light source configured to emit excitation light for exciting advanced glycation end products generated by performing a heat treatment on a target region of a biological tissue;
an imaging device including an imaging element configured to generate a first fluorescence image obtained by imaging the target region by imaging fluorescence emitted by the excitation light and a second fluorescence image obtained by imaging the target region after a time at which the first fluorescence image is captured; and
a medical device comprising a processor comprising hardware, the processor being configured to
acquire the first fluorescence image and the second fluorescence image,
generate a drive signal for causing a display device to display discharge information indicating a discharge status of a resected piece resected by a resection treatment tool in the target region based on first information included in the first fluorescence image and second information included in the second fluorescence image, and
output the drive signal.

8. The medical system according to claim 7, further comprising a perfusion device configured to supply a perfusate toward the target region,
wherein the drive signal includes a signal for controlling the perfusion device.

9. A method of operating a medical device including a processor and driven according to a cleaning state of a target region, the method comprising:
acquiring, by the processor, a first fluorescence image obtained by imaging the target region and a second fluorescence image obtained by imaging the target region after a time at which the first fluorescence image is captured;
generating, by the processor, a drive signal for causing a display device to display discharge information indicating a discharge status of a resected piece resected by a resection treatment tool in the target region based on first information included in the first fluorescence image and second information included in the second fluorescence image; and
outputting, by the processor, the drive signal.

10. A non-transitory computer-readable recording medium with an executable program stored thereon, the program causing a processor of a medical device driven according to a cleaning state of a target region to execute:
acquiring a first fluorescence image obtained by imaging the target region and a second fluorescence image obtained by imaging the target region after a time at which the first fluorescence image is captured;
generating a drive signal for causing a display device to display discharge information indicating a discharge status of a resected piece resected by a resection treatment tool in the target region based on first information included in the first fluorescence image and second information included in the second fluorescence image; and
outputting the drive signal.
